(19) United States
(12) Patent Application Publication
(10) Pub. No.: US 2026/0093944 A1
(43) Pub. Date: Apr. 2, 2026

(54) MEDICAL DEVICE HAVING A TEMPERATURE SENSITIVE ACTIVATION LABEL

(71) Applicant: Roche Diabetes Care, Inc., Indianapolis, IN (US)

(72) Inventor: Kirill Sliozberg, Mannheim (DE)

(21) Appl. No.: 19/410,489

(22) Filed: Dec. 5, 2025

Related U.S. Application Data
(63) Continuation of application No. PCT/EP2024/065554, filed on Jun. 6, 2024.

(30) Foreign Application Priority Data
Jun. 9, 2023 (EP) 23 178 437.2

(51) Int. Cl. G06K 7/14; A61M 5/172

(52) U.S. Cl. CPC G06K 7/1417; A61M 5/1723; G06K 7/1413; A61M 2205/0238; A61M 2205/60; A61M 2205/6063

(57) ABSTRACT

A medical device has a medical tool and an activation label connected to the medical tool. The activation label has an activation code required for activating the medical device. A controller is connected to the medical tool and the controller is configured to receive an activation signal from a remote control device. The activation label has a temperature sensitive part that is configured to change appearance at a predetermined temperature condition, in particular such that it masks at least part of the activation code if the predetermined temperature condition is reached.

### RELATED APPLICATIONS

This application is a continuation of International Application No. PCT/EP2024/065554, filed Jun. 6, 2024, which claims priority to EP 23 178 437.2, filed Jun. 9, 2023, the entire disclosures of both of which are hereby incorporated herein by reference.

### BACKGROUND

This disclosure relates to a medical device having an activation label, a medical device activation system, a server for enabling activation of a medical device, a method of activating a medical device and the use of a remote control device to activate a medical device as well as the use of a medical device activation system or a server for enabling activation of a medical device.

It is known that medical devices, in particular those containing chemical and/or biochemical compounds, e.g., proteins, and/or synthetic substances and/or or small structures, e.g., septa, may be temperature sensitive, i.e., require a handling and storage in a certain temperature range and can be damaged when stored or handled outside of said temperature range, in particular at high temperatures. A handling at too high temperatures may affect their ability to function properly, e.g., reduce or destroy their ability function. In the case of a sensor using chemical and/or biochemical compounds for sensing an analyte, the calibration may be destroyed or the sensor may even be rendered unusable by handling outside of the acceptable temperature range. This may, however, not be visible from the medical device itself. Thus, a user may come to harm by relying on a medical device if during handling or storage the medical device has been exposed to temperatures outside of the acceptable temperature range.

Attempts to overcome this problem have been made. For example, U.S. Pat. No. 6,892,085 B2 discloses a glucose sensor package system, EP 3 787 479 A2 discloses an automatic analyte sensor calibration and error detection; U.S. Publication No. 2022/0192597 A1 discloses systems and methods for analyte detection and U.S. Publication No. 2022/095968 A1 discloses systems, devices and methods to compensate for temperature effects on sensors. Nevertheless, it remains an issue how to reliably protect a user from potentially damaged medical devices.

### SUMMARY

This disclosure relates to the problem of reliably protecting a user from potentially damaged medical devices.

This disclosure teaches a medical device comprising a medical tool. The medical tool may in particular comprise one or more temperature sensitive parts or components. The medical tool may, for example, be or comprise a physiological sensor or a device for administering a drug. It may be a medical tool for insertion into living tissue, e.g., for subcutaneous insertion. The medical tool may in particular be an analyte sensor, in particular for sensing a particular analyte or concentration of an analyte, e.g., when inserted subcutaneously. The analyte may e.g., be glucose or alcohol or a virus or a hormone or dopamine or ketones, the analyte is in particular glucose. Alternatively, the medical tool may be a device for administering a drug, which comprises a temperature sensitive part or component, e.g., the drug, and/or another temperature sensitive part. In particular, the drug is a liquid drug, preferably insulin.

The medical device may, for example, comprise a housing in which parts of the medical tool and/or the controller as well as any optionally present other parts, e.g., batteries, may be arranged. In particular, the medical device may, e.g., be a continuous glucose monitoring system, in particular comprising a glucose sensor, or a continuous insulin delivery device, e.g., a continuous subcutaneous insulin infusion device. The medical device may comprise a patch for affixing the medical device to a user. The medical device may comprise a housing. The housing may, for example, be arranged on top of at least part of the patch of the medical device, i.e., on the side of the patch, which, in use, is opposite to the user's skin. Thus, the patch may in particular also affix the housing to the user. The medical device may be delivered in an applicator that serves as help in applying the medical device to a user, in particular the applicator is configured for inserting the medical tool into a tissue, in particular into the skin of a user, and which may be removed after the medical device is applied for use.

The medical device comprises an activation label connected to the medical tool. This connection may be direct, e.g., by the label being arranged on the medical tool, or indirect, e.g., by the activation label being arranged on or comprised by a part of the medical device that is (directly or indirectly) connected to the medical tool, or being otherwise connected, e.g., affixed or connected by a packaging with the medical device. The connection between the activation label and the medical tool has in particular the purpose to make sure the medical device and activation label are not separated to far from each other. In particular, this connection thus ensures that the medical device, in particular the medical tool and the activation label are exposed to the same temperature condition during storage and handling.

The activation label may be in the form of a sticker, printing, or a coating on a piece of the medical device or a piece attached to it, or in other forms.

The medical device further comprises a controller connected to the medical tool, which is configured to receive an activation signal from a remote control device. In particular the controller can be electrically connected and/or mechanically connected to the medical tool. The controller may, in particular, have its own energy source, e.g., a small battery, in particular button cell, an antenna, and a communication interface for communicating with a remote control device, e.g., by WiFi, Bluetooth, NFC or some other wireless communication protocol. Alternatively, the communication interface may communicate with the remote control in a wired fashion. The controller may also comprise a memory for storing information.

The controller is configured to activate the medical tool, and hence activate the medical device, when it receives an activation signal from a remote control device. The activation label in particular comprises an activation code required for activating the medical device, in particular for starting its functioning, e.g., the measurement in the case of a sensor or the pumping of the drug in the case of a device for administering a drug. In particular, the medical device is configured that it cannot be used without activation, in particular not without determination and processing of the activation code. The activation signal from the remote control device may, for example, be based on the activation code. In particular, it may be calculated or otherwise based on the activation code, or be looked up in a list based on the activation code or be or comprise the activation code. The activation signal may, for example, only be generated after a verification step of the activation code has been carried out, e.g., by the remote control device, and/or the device may only be activated after a verification step of the activation signal and/or the activation code has been carried out, e.g., by the controller.

The activation label comprises a temperature sensitive part that is configured to (irreversibly) change its appearance at a predetermined temperature condition. The change of appearance may in particular be such that it masks at least part of the activation code, in particular the entire activation code, if the predetermined temperature condition is reached. Thus, activation of the device may be different or not possible at all, when a predetermined temperature condition is reached during transport or storage of the medical device, which is an advantage that protects a user from using a medical device which may be damaged when it is stored at certain temperature conditions.

A predetermined temperature condition may in particular be that a) a predetermined temperature is reached or exceeded, b) a predetermined temperature is reached or fallen below, c) a predetermined temperature has been reached or exceeded for a certain amount of time, d) a predetermined temperature has been reached or fallen below for a certain amount of time, or e) the temperature has exceeded a predetermined temperature while at the same time a predetermined higher temperature is not reached, once or for a certain time.

The predetermined temperature conditions, in particular the predetermined temperatures, are usually temperature conditions, in particular temperatures, that depend on the medical device, in particular the medical tool. Typically, the predetermined temperature condition, in particular temperature, is a threshold at which the properties of the medical tool change, e.g., render it less or more sensitive for the quantitative detection of an analyte in the case of a sensor, or render it less or more specific for the qualitative detection of an analyte in the case of a sensor, hence leading to an erroneous measurement of the determined analyte, or render it unusable or unreliable. These thresholds are typically known; for example, they can be determined in advance by testing the medical devices under certain known and determined environment conditions, in particular temperature conditions. Thus, the change of appearance of the activation label may be indicative of a change of a property of the medical device, in particular of the medical tool. In particular the change in property can be a change of integrity of functioning and/or change of safety of the use of the medical device. Based on the described testing the predetermined temperatures and storage time intervals can be determined that do or do not adversely affect the integrity of functioning and/or safety of the use of the medical device.

The change of appearance may in particular mask at least part of the activation code, in particular the entire activation code, if the predetermined temperature condition is reached, e.g., by taking the same appearance as part of the activation code, or hiding part of the activation code in another manner, thus rendering it unreadable. Thus, a user may not use at least part of the activation code, in particular the entire activation code, to activate the medical device once the predetermined temperature condition has been reached, e.g., during storage, handling or transport. In particular, if part of the activation code, in particular the entire activation code, is masked, a user will be restricted in activating the medical device, in particular unable to activate the medical device, and hence may in particular be efficiently and be advantageously protected from using a product whose integrity of functioning and/or safety of use has been compromised due to having been stored or transported out of the acceptable temperature range.

The activation label may in particular comprise a coating of a temperature sensitive material, in particular of temperature sensitive ink, that has locally been treated with the predetermined temperature condition, e.g., locally heated with or above a predetermined temperature, in particular treated with the predetermined temperature condition for a predetermined time interval, to display the activation code. The coating of temperature sensitive material may be applied on the medical device, e.g., the housing or the patch, by some kind of coating or printing, e.g., using screen printing. In particular, by treatment with the predetermined temperature condition, e.g., through a laser, in particular with a low-power, the coating of the temperature sensitive material may be treated such that it changes its appearance to display the activation code. In this manner, the activation code may be written onto the temperature sensitive material. When the part of the temperature sensitive material that has not been treated with the predetermined temperature condition to display the activation code is exposed to the predetermined temperature condition, it will take the same appearance as the activation code, thus masking it, in particular hiding it from view.

Such temperature sensitive materials, in particular temperature sensitive inks, are commercially available. These temperature sensitive materials may, in particular, be phase-change indicators, which employ a specifically purified organic compound with a sharply defined melting point and high-precision color change effect. Substances with different trigger temperatures are available, starting as low as 25° C., and having a precision of up to 1° C. Additionally, different compositions allow choosing whether there is an immediate response to reaching a certain temperature or whether the response happens slowly when the temperature is reached, thus providing a change only when a predetermined temperature is reached for at least a certain time. In particular, the temperature sensitive materials may thus be tailored to the predetermined temperature condition depending on the temperature sensitivity of the medical device, in particular the medical tool. An example for such a temperature sensitive material is thermochromic ink NH SDS004 Rev4 by LCRHallvcrest (https://spotsee.io/wp-content/uploads/2021/05/PDS078-Rev01.pdf).

The needed energy input to change the appearance of the temperature sensitive material may in particular depend on the particular properties of the used temperature sensitive material, in particular temperature sensitive ink, in particular its heat capacity. The heat capacity, usually measure din J/g indicates, how much energy has to be introduced in 1 g of the temperature sensitive material to induce the color switch. Depending on the used temperature sensitive material, the layer thickness of the temperature sensitive material, the area of the modified surface and on the power dissipation properties of the underlying material, the power of a writing laser may be adjusted to treat the temperature sensitive material such that it changes it appearance when writing the activation code. Additional parameters that may influence the necessary power of the laser may include the ink absorbance for the given laser irradiation and/or the ambient temperature (and thus in particular the temperature of the surface to be modified). In particular, the smaller the designed heat capacity is, the more sensitive the ink is for temperature changes. As an example, with a typical heat capacity of temperature sensitive inks in the range 10-500 J/g, an exemplary ink layer of 5 μm thickness and a single laser spot of 1 mm in diameter, this yields a needed input power 40-2000 μJ in case of 100% laser irradiation absorbance and no heat dissipation by underlying material. Such low-power laser irradiation may, for example, be achieved by applying of ultra-short laser pulses. Since there are many variables defining the needed laser input power, laser power calibration may in particular be performed using test structures before each lot of products is modified, in particular by writing at least a part of the activation code onto a temperature sensitive material using a laser.

The temperature sensitive part may be configured to changes its appearance (from an original appearance) to a first appearance at a first predetermined temperature condition and to change its appearance to a second appearance, that is in particular different from the first appearance, at a second predetermined temperature condition, that is in particular different from the first predetermined temperature condition. For example, a first part of the activation code may optionally be displayed due to a temperature sensitive material having been locally treated with the first predetermined temperature condition to display the activation code, and a second part of the activation code may optionally be displayed due to a temperature sensitive material having been locally treated with the second predetermined temperature condition. Other ways of displaying the first and/or second and/or further parts of the activation code, e.g., by normal printing in a color that can be masked by a temperature sensitive part after its change of appearance, are also possible. In particular, the change to the first appearance may result in masking (hiding) at least a first part of an activation code and the change to the second appearance masking (hiding) at least a second part of the activation code, in particular masking (hiding) the entire activation code.

The temperature sensitive part may in particular be configured to change its appearance several, e.g., three, four or more, times at several, e.g., three, four or more, temperature conditions (which are in particular each distinct from all other temperature conditions), each change of appearance optionally masking a (further) part of the activation code. Each part of the activation code may optionally be displayed (before it is masked) due to a temperature sensitive material having been locally treated with one of the several predetermined temperature conditions to display the activation code. For example, a third part of the activation code may optionally be displayed due to a temperature sensitive material having been locally treated with a third predetermined temperature condition to display the activation code. Optionally, the temperature sensitive part may be configured to change its appearance to a third appearance (different from the first and second appearance) at the third predetermined temperature condition (different from the first and second temperature condition), masking at least a third part of the activation code.

With a partially masked activation code, activation of the medical tool may still be possible. The remaining part(s) of the activation code that is/are still visible may then be used to activate the medical device with one or more particular start parameters and/or calibration. Such one or more particular start parameters or calibration may in particular be or comprise that the device can still start, but the controller provides the measurement data in a manner such that user is warned of lower measurement precision and/or the device operation time may be limited, in particular by the controller, to a particular duration, as a medical device, in particular medical tool, that has been stored at temperatures higher than allowed may show signal drift earlier than one that has been properly stored. In particular, in a system that provides measurement data to the user, e.g., by delivering the same to a screen of the remote control device, the user may, e.g., be warned of lower measurement precision by delivering the measurement data with lower resolution and/or higher error bars.

The last (e.g., the second, or third or a further) of the several changes of appearances may in particular mask the entire activation code such that an activation of the medical device is not possible anymore. The first, second, and optionally third and further temperature conditions may, e.g., each correspond to reaching first, second, optionally third and further temperatures, wherein each temperature is higher than the previous one. Thus, when the last (in this example highest) temperature is reached, at which the device may be too damaged to be safely usable, it may be impossible to activate the device.

The temperature sensitive part may, e.g., be configured such that it comprises two or more parts, each of a different temperature sensitive material (in particular temperature sensitive materials displaying a change of appearance at one of the predetermined temperature conditions) arranged close to each other (e.g., both visible at the same time), next to each other and/or on top of each other. Additionally or alternatively, the temperature sensitive part may comprise one or more temperature sensitive materials that may show two or more changes of appearances at different temperatures conditions, optionally arranged close to, next to or on top of one or more other temperature sensitive materials showing one or more changes of appearances. This way, different parts of the activation code may be written next to each other on different parts of the activation label and/or may be written in overlapping areas.

The activation label may in particular be or comprise a coating of a temperature sensitive material, in particular of temperature sensitive ink that is configured to change its appearance to a first appearance, e.g., a first optical density, at a first predetermined temperature condition and to change its appearance to a second appearance (which is in particular different from the first appearance), e.g., a second optical density, at a second predetermined temperature condition (wherein the second predetermined temperature condition is in particular different from the first predetermined temperature condition). Such temperature sensitive materials, in particular inks, may have two or more predetermined temperature conditions at which they change their appearance, e.g., their optical density.

This may, in particular, allow for a first change of appearance to indicate a first event of the medical device, in particular the medical tool, e.g., a change, e.g., decrease or increase, in sensitivity, and allow the second change of appearance to indicate a second even of the medical device, in particular the medical too, e.g., it being now being unusable.

In particular, by providing the activation code in two different appearances, e.g., a first part of the activation code being indicated in a first appearance corresponding to the first change of appearance (of the activation label) and a second part of the activation code being indicated in a second appearance corresponding to the second change of appearance, this may lead to an activation code that is partially masked, in particular partially hidden, at the first predetermined temperature condition. The remaining part of the activation code that is visible may then be used to activate the medical device with one or more particular start parameters or calibration. If the second predetermined temperature condition is reached, the activation code may then be fully masked, in particular fully hidden, such as to prohibit an activation of the medical device.

The activation label may comprise a first temperature sensitive part configured to change its appearance at a first predetermined temperature condition and a second temperature sensitive part configured to change its appearance at a second predetermined temperature condition (which is in particular different from the first predetermined temperature condition). This may, for example, be provided by coating two different temperature sensitive inks next to each other or onto each other. This may, in particular, also allow to provide an activation code in two different appearances, wherein one appearance is masked at a first predetermined temperature condition and a second appearance is masked at a second predetermined temperature condition.

The activation code may also comprise more than two, e.g., three, four or five different appearances, in particular wherein the additional appearances each may optionally be reached at a predetermined temperature condition (which is in particular different from the other predetermined temperature conditions by which other appearances are reached), in particular a (or each) predetermined temperature condition corresponding to a temperature threshold changing the medical device. The activation label may be configured to display the entire activation code (or all parts thereof) in a manner that it (or a part thereof) will be masked when a (each) predetermined temperature condition is reached.

The activation label may in particular be arranged hidden from view of a user before unpacking of the medical device, in particular before application of the medical device. For example, the activation label may be arranged inside a packaging in a manner that is not readable from the outside, in particular hidden from the view of a user prior to unpacking of the medical device and/or application of the medical device, in order to avoid that a user copies the activation code or activates the medical device before actually using the medical device only at a later point in time when the medical device has reached a predetermined temperature condition which, e.g., compromise the integrity or safety of use of the medical device. Such behavior would bypass the safety function of the activation label. If the medical device comprises a housing, the activation label may in particular be arranged on a side or part of the housing that is not visible and/or accessible to a user before the medical device is removed, thus allowing the user to determine the activation code once the packing has been removed. In particular, if the medical device is delivered with an applicator, the activation label may be arranged inside the applicator, such that it is only visible and/or accessible to the user after the medical device has been applied with the applicator. Alternatively, the activation label may be arranged on the patch such that it is only visible and/or accessible to the user after the medical device has been applied and the applicator is removed thereof. In view of the above, this disclosure is in particular associated with the advantage of increasing the safety of use of the medical devices of this disclosure.

In particular, if the activation code is hidden from view of the user before the unpacking, in particular before application of the medical device, the packaging of the medical device may comprise a label, in particular a label of a temperature sensitive material, indicating whether the predetermined temperature condition has been reached. This label comprised by the packaging of the medical device may not display the activation code, but may enable the user to determine before application of the medical device whether or not the predetermined temperature condition has been reached, thus avoiding an application of a system that does not function properly. Nevertheless, through the application code it is made sure that even if the user misses the indication, a faulty medical device will not be used.

The activation code or parts thereof may be displayed in letter and digits, in particular comprise letters and/or digits, and/or as a QR-code. Alternatively or additionally, the activation code or parts thereof may be displayed as a bar code or in some other, in particular machine readable, format.

This disclosure further comprises a medical device activation system comprising the medical device as described and a remote control device configured to determine information allowing determination of the activation code from the activation label and send an activation signal to the medical device based on the activation code. In particular, the remote control device may be a cellphone, e.g., with an appropriate app for controlling the medical device, a computer, tablet or laptop, also, e.g., with an appropriate app for controlling the medical device. The remote control device may have a part allowing to determine information allowing determination of the activation code from the activation label. Information allowing determination of the activation code may in particular be the activation code itself or an image of the activation label or some other information allowing determining of the activation code. The part allowing determining of information allowing the determination of the activation code may, e.g., be or comprise an interface, into which the activation code can be typed in, a camera to scan the activation code or take a photo of the activation label, in particular the activation code, and optionally process it by image processing, or some other way of determining information allowing determination of the activation code. In particular, the activation code may be determined from the activation label by optical scanning, in particular optical scanning with a camera, e.g., a camera of a cell phone, computer, tablet or laptop. The remote control device may then send an activation signal to the medical device based on the activation code. The activation signal may be calculated or determined by the remote control device, or be received by the remote control device, e.g., from another source, and be forwarded to the medical device. If no activation code can be determined from the activation label, no activation signal may be sent to the medical device, and thus use thereof may not be possible.

In particular, the remote control device may be a handheld device. The remote control device may possess means for reading or determining the activation code, in particular directly or indirectly read or determine it, e.g., a camera or scanner. The remote control device may be a remote control device only usable for remote control of the medical device or may only be useable for control of medical devices, or may alternatively also be used for other applications.

In particular, the medical device activation system may further comprise a processor configured to receive the information allowing determination of the activation code, in particular the activation code, e.g., (directly) from the remote control device or from a part of the remote control device, e.g., a camera or part having carried out a calculation or image processing to determine the activation code. The processor may further be configured to determine the activation code from the information allowing determination of the activation code, e.g., if the information allowing determination of the activation code is not the activation code itself. The processor may further be configured to carry out a verification step of the activation code, in particular compare it with a reference activation code. The processor may be configured to generate a confirmation signal to allow activation of the medical device, in particular if the verification step is successfully. The confirmation signal to allow activation of the medical device may in particular be the activation signal or comprise information necessary to allow the remote control device or parts thereof to determine the activation signal. The processor may be configured to forward the confirmation signal to allow activation of the medical device to the remote control device or to a part of the remote control device, e.g., the app, configured to send the activation signal to the medical device based on the activation code. The processor may also be configured to, if the verification step is not successful, not send a confirmation signal to allow activation of the medical device.

The processor may be part of the remote control device, and reference activation codes may, e.g., be stored on the remote control device. Such reference activation codes may, e.g., be calculated, received by download from a further source, e.g., a server, or be provided to the remote control device in some other manner. The reference activation codes may be downloaded or updated regularly. Alternatively, the processor may be comprised by a remote server or computer, e.g., a server of the provider of the medical device. In particular, carrying out the verification step by a remote server may allow some counterfeit protection by using individual activation codes only once and/or may allow reacting to additional information, e.g., errors in production charges, e.g., by removing those verification codes from the reference verification codes, in particular such that the verification of any medical device of said charge cannot be successful. The processor may be configured to not send a confirmation signal to allow activation of the medical device if the verification step is not successful. Then, in particular, no activation signal can hence be sent to the controller by the remote control device. Alternatively, the processor may be split between the remote control device and a server, such that parts of the steps described above for the processor are carried out by a processor on the remote control device and some, in particular the others, are carried out by a process on a (remote) server.

This disclosure further comprises a server for enabling activation of an above-described medical device. The server may in particular be configured to receive information allowing determination of the activation code from the remote control device and send an activation confirmation signal to the remote control device or a part thereof to allow activation of the medical device. The activation confirmation signal may in particular be the confirmation signal to allow activation of the medical device mentioned before. Communication between the server and the remote control device may, e.g., be wireless or wired communication over the internet, e.g., by e-mail, FTP, TCP, or other suitable formats. The server may optionally be configured to determine the activation code from the information allowing determination of the activation code, e.g., if the information allowing determination of the activation code does not explicitly indicate the activation code. The server may further optionally be configured to carry out a verification step of the activation code, e.g., as described above for the processor. The server may be the remote server comprising the above-mentioned processor and/or be configured to carry out one or more of the functions for which the processor described above is configured.

This disclosure further comprises a method of activating a medical device, in particular a medical device as described above, comprising determining information allowing determination the activation code. This may, e.g., comprise scanning the activation label in order to determine the activation code from the activation label, and may in particular comprise scanning the activation code on the activation label. The method may further comprise carrying out a verification step of the activation code, in particular comparing it with a reference activation code. The method further comprises sending an activation signal to the medical device based on the activation code, in particular if the verification step is successful. If the verification step is not successful, no activation signal may be sent to the medical device. The method steps may be carried out by the same entity, e.g., a remote control device, or be carried out by different entities, e.g., the first by a remote control device, the second by a remote server and the third again by the remote control device. Other combinations are possible as well. The method may in particular comprise the steps described above with regard to the medical device.

This disclosure further comprises the use of a remote control device to activate a medical device as described above. The use may, in particular, comprise determining, in particular scanning, information allowing determination of the activation code from the activation label. The use may in particular comprise the remote device determining, in particular scanning, the activation label, in particular the activation code. The use in particular comprises sending an activation signal to the medical device based on the activation code. The use may also comprise the other steps that may be carried out by the remote control device outlined above.

This disclosure also comprises the use of a medical device activation system as described above and/or the use of a server for enabling activation of medical device as described above in order to activate the medical device. The use may in particular comprise all steps outlined above with regard to the medical device and/or the medical device activation system and/or the server for enabling activation of a medical device.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is an image showing color density versus temperature of two exemplary temperature sensitive inks;

FIG. 2 shows an exemplary medical device;

FIG. 3a shows an exemplary activation label;

FIG. 3b shows the activation label of FIG. 3a after it has been exposed to the predetermined temperature condition;

FIGS. 4a and 4b show exemplary steps in generating an activation label;

FIG. 4c shows the exemplary activation label after a first predetermined temperature condition has been reached;

FIG. 4d shows the exemplary activation label after a second predetermined temperature condition has been reached;

FIG. 5 shows exemplary steps of a method; and

FIG. 6 is a schematic drawing of an exemplary medical device activation system.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

FIG. 1 shows color density vs. temperature of two exemplary temperature sensitive inks; for both inks, the opaqueness increases with temperature. The first one (continuous line) has a rather sharp increase of the optical density (e.g., for more than 0.5, e.g., more than 0.75, e.g., more than s1) in a narrow range, e.g., in less than 25, e.g., less than 10° C., then remaining at rather similar optical densities (e.g., a change of no more than 0.5) for a further range of higher temperatures, e.g., for more than 40° C. The second ink (the data of which is indicated in a dotted line) has a less steep, but rather continuous increase in optical density with increasing temperature. Each of these inks alone or both of these inks may be used to for the temperature sensitive part of an activation label.

The increase of the opaqueness with the temperature in particular allows to create above-described activation labels. The first ink may in particular be used for generating an activation label displaying an activation code and masking the entire activation code when a predetermined temperature condition is reached, in particular once a threshold temperature is reached.

The second ink (indicated in a dotted line) may in particular be used for comprising two or more appearances (i.e., optical densities) in one activation label, such that a first part corresponding to the first appearance of the activation code becomes masked when a first predetermined temperature condition is reached, e.g., a first threshold temperature is reached, and a second part of the activation code corresponding to the second appearance of the activation code becomes masked when a second predetermined temperature condition, e.g., a second threshold temperature, is reached.

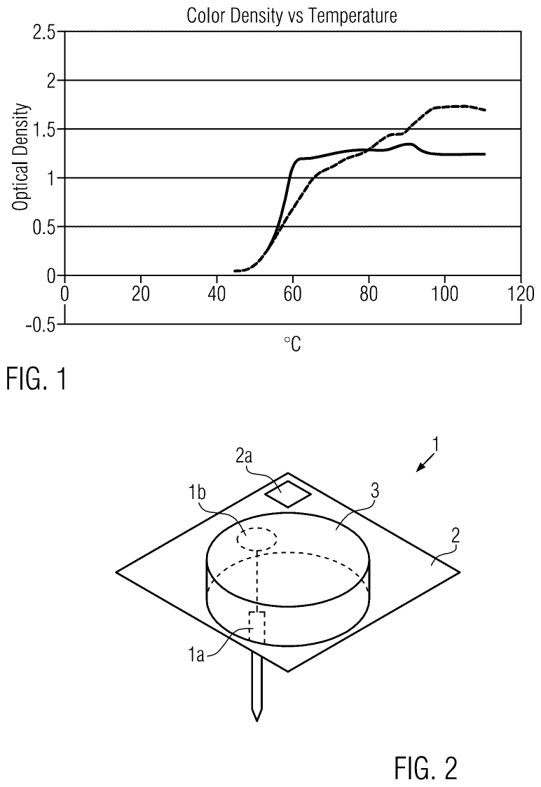

FIG. 2 shows an exemplary medical device 1 (as it would be schematically seen when applied to a user). The medical device 1 may be applied using an applicator (not shown). The medical device comprises a medical tool 1a and a controller 1b and optionally comprises a housing 3. In the exemplary medical device 1, which may in particular be a CGM system (continuous glucose monitoring system), the medical tool 1a, e.g., a glucose sensor allowing CGM, is connected to the controller 1b. This connection is schematically indicated by a line. The medical tool may in particular be a medical tool for subcutaneous insertion. The medical device 1 may further comprise a patch 2, which may be configured to attach the medical tool 1a and controller 1b to a user when the medical tool 1a is used. In particular, the medical tool 1a may extend through patch 2, thus allowing to be, in particular subcutaneously, inserted into the skin of a user, while the part of the medical tool 1a extending on the other side of the patch may be connected to the controller 1b. Both the controller 1b and the part of the medical tool 1a on the side of the patch 2, which in use is not on the skin of a user, may be arranged in the housing 3. In other embodiments, only one or neither may be arranged in a housing (not shown). The medical device 1 may be delivered arranged in the applicator (not shown), and can be applied to the body of a user with the housing 3. After the patch 2 is applied, the applicator may be removed. The activation label may in particular be arranged as coating, e.g., on the inside of the applicator or on a part of the housing covered by the applicator or on a part of the patch (here exemplarily indicated by 2a), which may in particular be covered by the applicator before the applicator is removed, such that the activation label, in particular the activation code, only becomes visible after the patch 2 has been applied to the user.

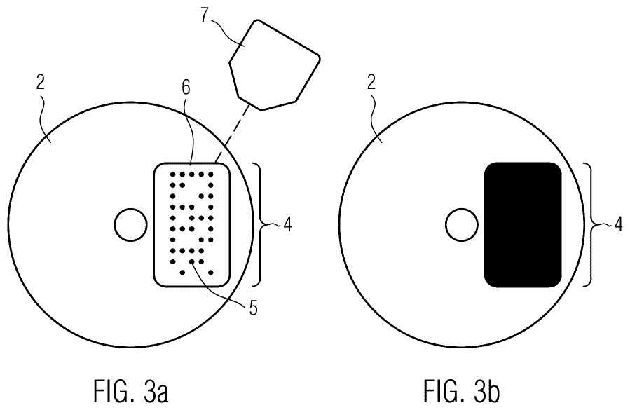

FIG. 3a shows an exemplary activation label 4 comprising an activation code 5, here exemplarily on a round patch 2, which may be comprised by the medical device 1. In other embodiments, such an activation label 4 may be arranged on a patch in another form or other parts of the medical device (not shown). The activation code 5 is exemplarily provided in the form of a QR code. This activation label may, e.g., be arranged inside the housing or on a patch, or be connected to the medical tool in another manner. The activation label 4 in particular comprises a temperature sensitive coating 6, here exemplarily on a patch 2, that has locally been treated with the predetermined temperature condition in order to display the activation code 5. Such a treatment may, for example, be carried out with a laser 7, e.g., at low power (the laser 7 is not part of the medical device, but may be used in generating the activation code 5).

FIG. 3b shows the exemplary activation label of FIG. 3a after the entire activation label 4 has been exposed to a predetermined temperature condition. By this, the other parts of the activation label 4 are also changed as the area where the activating code was previously displayed, thus masking, in particular hiding, the activation code. Because the medical tool is connected to the activation label during transport and storage thereof, any temperature the medical tool reaches is also reached by the activation label 4. Hence, if the predetermined temperature condition that is known to render the medical device 1, in particular the medical tool, unusable, the activation code is masked, in particular hidden, and hence cannot be determined to allow activation of the medical device.

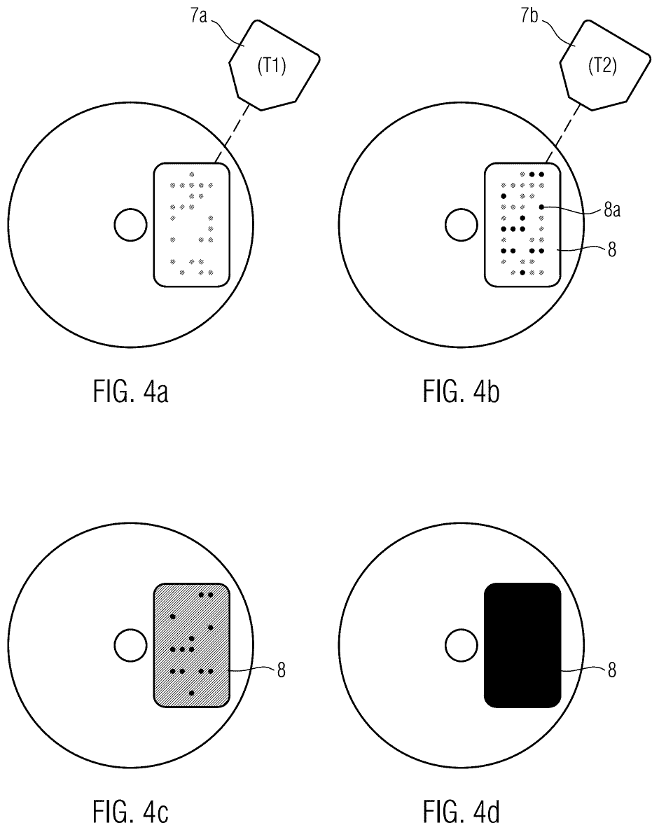

FIG. 4a shows a step in the generating an exemplary activation label. The exemplary activation label may, in particular, be arranged on part of the medical device, e.g., the patch as indicated here as an example. The exemplary activation label is generated by first treating a temperature sensitive coating locally with a first predetermined temperature condition, in particular a first temperature T1. The coating may have been applied using some printing or other coating technique.

FIG. 4b shows the exemplary further step in generating of an exemplary activation label 8, where the temperature sensitive coating of FIG. 4a is locally treated with a second predetermined temperature condition T2. The treating of the temperature sensitive coating may exemplarily both times be carried out using lasers 7a, 7b. However, other ways of treating the coating with temperature conditions may also be used (not shown). The temperature T2 may be higher (or lower) than temperature T1. Through the generation steps, an activation label 8 is generated from the temperature sensitive coating which has an activation code 8a with a first part with a first appearance (indicated in grey) and a second part with a second appearance (indicated in black).

FIG. 4c shows the exemplary activation label 8 of FIG. 4b after a first predetermined temperature condition, in particular first threshold temperature T1, has been reached by the label 8, thus changing the appearance of the temperature sensitive ink to mask the parts of the activation code having the first appearance, and only leaving readable the parts of the activation code having the second appearance.

FIG. 4d shows the exemplary activation label 8 of FIG. 4a, 4b after a second predetermined temperature condition, in particular second threshold temperature T2, has been reached. There, no part of the activation code can be read. Hence, activation of the medical device which comprises the activation label is not possible, and a user may in particular be protected from using a medical device that has been handled or stored in an unacceptable temperature range.

In other examples (not shown), the activation label may be configured to display more than two, e.g., three or more, changes of appearances at three (or more) predetermined temperature conditions, each change of appearance masking a further part of the activation code, wherein the last change of appearance masks the entire activation code. For example, for this a coating may be used that shows three such changes of appearance, or a further part of the activation code may be written on a coating displaying a change of appearance at a different predetermined temperature condition arranged next to or near the coating displayed in FIGS. 4a-4d and/or it may be arranged overlapping.

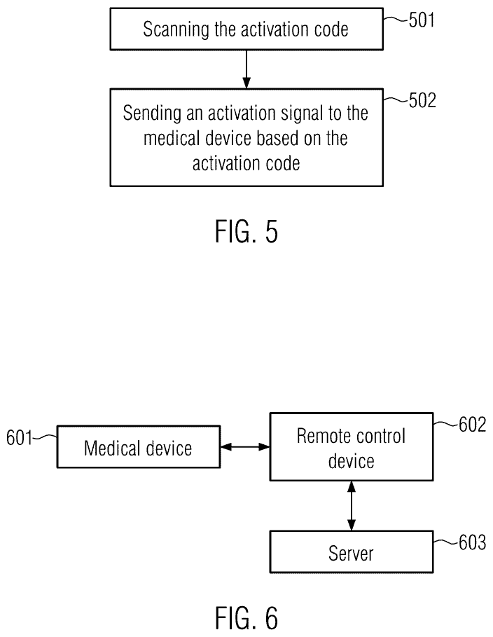

FIG. 5 shows exemplary steps of a method for activating a medical device. In particular, in step 501, the activation label is scanned, in order to determine information allowing determination of the activation code. In particular, in said step, the activation code may be scanned; then, in step 502, an activation signal is sent to the medical device, thus activating it for use. Without said activation signal the medical device cannot be used. In particular, if the medical device has been exposed during transport to unacceptable temperatures, the activation code cannot be determined as it is masked and the user is protected from using a damaged medical device.

FIG. 6 is a schematic drawing of an exemplary medical device activation system. The exemplary medical device activation system comprises a medical device 601, a remote control device 602 configured to send an activation signal to the medical device 601, and optionally also a remote server 603 configured to at least carry out a verification step of the activation code.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A medical device, comprising:
a medical tool;
a controller connected to the medical tool, the controller configured to receive an activation signal from a remote control; and
an activation label connected to the medical tool and having an activation code required for activating the medical device, wherein the activation label comprises a temperature sensitive part configured to change appearance at a predetermined temperature condition.

2. The medical device of claim 1, wherein the activation label comprises a coating of a temperature sensitive material locally treated with the predetermined temperature condition to display the activation code.

3. The medical device of claim 1, wherein the temperature sensitive part is configured to change appearance to a first appearance at a first predetermined temperature condition and to change appearance to a second appearance at a second predetermined temperature condition.

4. The medical device of claim 1, wherein the activation label comprises a coating of a temperature sensitive material that is configured to change appearance to a first appearance at a first predetermined temperature condition and to change appearance to a second appearance at a second predetermined temperature condition and/or wherein the activation label comprises a first temperature sensitive part configured to change appearance at a first predetermined temperature condition and a second temperature sensitive part configured to change appearance at a second predetermined temperature condition.

5. The medical device of claim 1, wherein the activation label is hidden from the view of a user before unpacking of the medical device.

6. The medical device of claim 1, wherein packaging of the medical device comprises a label indicating whether the predetermined temperature condition has been reached.

7. The medical device of claim 1, wherein the activation code is displayed in letters and digits, as a bar code, or as a QR-code.

8. The medical device of claim 1, wherein the medical tool is a physiological sensor and/or a medical tool for subcutaneous insertion.

9. A medical device activation system, comprising:
the medical device of claim 1; and
a remote control configured to determine information allowing determination of the activation code from the activation label and send an activation signal to the medical device based on the activation code.

10. A medical device activation system according to claim 9, wherein the information allowing determination of the activation code is determined from the activation label by optical scanning.

11. Use of the medical device activation system of claim 9 to activate the medical device.

12. The medical device activation system of claim 9, further comprising a processor configured to receive the information allowing determination of the activation code and generate a confirmation signal to allow activation of the medical device.

13. A server for enabling activation of the medical device of claim 1, the server being configured to receive information allowing determination of the activation code from a remote control, and send an activation confirmation signal to the remote control or a part thereof to allow activation of the medical device.

14. Use of the server for enabling activation of a medical device according to claim 13 to activate the medical device.

15. A method of activating the medical device of claim 1, the method comprising:
determining information allowing determination of the activation code; and
sending an activation signal to the medical device based on the activation code.

16. Use of a remote control to activate a medical device of claim 1.

17. The medical device of claim 1, wherein the temperature sensitive part is configured to mask at least part of the activation code if the predetermined temperature condition is reached.

18. The medical device of claim 17, wherein the temperature sensitive part is configured to entirely mask the activation code if the predetermined temperature condition is reached.

19. The medical device of claim 17, wherein the temperature sensitive part is configured to irreversibly mask at least part of the activation code if the predetermined temperature condition is reached.
